(19) United States
(12) Patent Application Publication
(10) Pub. No.: US 2023/0402192 A1
(43) Pub. Date: Dec. 14, 2023

(54) METHODS AND APPARATUS FOR IDENTIFYING HIGH-RISK PATIENTS FOR PROTECTED PERCUTANEOUS CORONARY INTERVENTIONS

(71) Applicant: Abiomed, Inc, Danvers, MA (US)

(72) Inventors: Dawn Bardot, Danvers, MA (US); Daniel Schwartz, Danvers, MA (US); Daniel Poscover, Newton Centre, MA (US); Daniel Housman, Newton Centre, MA (US)

(73) Assignee: Abiomed, Inc., Danvers, MA (US)

(21) Appl. No.: 18/332,050

(22) Filed: Jun. 9, 2023

Related U.S. Application Data
(60) Provisional application No. 63/351,028, filed on Jun. 10, 2022.
(60) Provisional application No. 63/352,407, filed on Jun. 15, 2022.
(60) Provisional application No. 63/425,553, filed on Nov. 15, 2022.

(51) Int. Cl. G16H 50/70; G16H 10/60; G16H 15/00; G16H 50/50

(52) U.S. Cl. CPC G16H 50/70; G16H 10/60; G16H 15/00; G16H 50/50

(57) ABSTRACT

Methods and apparatus for identifying whether a patient is eligible for a Protected Percutaneous Coronary Intervention (Protected PCI®) are provided. The method comprises receiving medical information for a patient, extracting one or more features from the received medical information, classifying the patient with regard to the patient's eligibility for a Protected PCI® based, at least in part, on the extracted one or more features, to generate a patient classification, and outputting an indication of the patient classification.

### RELATED APPLICATIONS

This application claims the benefit under 35 U.S.C. § 119(e) to U.S. Provisional Patent Application No. 63/351,028, filed Jun. 10, 2022, and titled “METHODS AND APPARATUS FOR IDENTIFYING HIGH-RISK PATIENTS FOR PROTECTED PERCUTANEOUS CORONORY INTERVENTIONS,” and claims priority to U.S. Provisional Patent Application No. 63/352,407, filed, Jun. 15, 2022, and titled “METHODS AND APPARATUS FOR IDENTIFYING HIGH-RISK PATIENTS FOR PROTECTED PERCUTANEOUS CORONORY INTERVENTIONS,” and to U.S. Provisional Patent Application No. 63/425,553, filed Nov. 15, 2022, and titled “METHODS AND APPARATUS FOR IDENTIFYING HIGH-RISK PATIENTS FOR PROTECTED PERCUTANEOUS CORONARY INTERVENTIONS,” the contents of each of which are incorporated herein by reference in their entirety.

### FIELD OF INVENTION

This disclosure relates to identifying patients for protected percutaneous coronary interventions.

### BACKGROUND

Percutaneous coronary interventions (PCIs) are minimally invasive procedures that are used to open blocked coronary arteries. Examples of PCIs include balloon angioplasties, angioplasties with a stent, and atherectomies. Patients with blocked coronary arteries may be candidates for PCI with some patients qualifying for a regular PCI, other patients qualifying for high risk PCI depending on various risk factors, and yet other patients not qualifying for high risk PCI owing to major risk factors.

### SUMMARY

Described herein are systems and methods for identifying patients that are at a high risk for complications and may benefit from mechanical circulatory support during percutaneous coronary interventions (PCI).

In some embodiments, a method of identifying whether a patient is eligible for a Protected Percutaneous Coronary Intervention (Protected PCIS) is provided. The method comprises receiving medical information for a patient, extracting one or more features from the received medical information, classifying the patient with regard to the patient's eligibility for a Protected PCI® based, at least in part, on the extracted one or more features, to generate a patient classification, and outputting an indication of the patient classification.

In one aspect, the medical information for the patient includes one or more of an electronic health record, a laboratory report, an electrocardiograph report, and a medical imaging report. In one aspect, the medical information includes structured data and unstructured data. In one aspect, extracting one or more features comprises extracting from the unstructured data, at least some of the one or more features using natural language processing. In one aspect, extracting one or more features comprises extracting at least some of the one or more features using natural language processing. In one aspect, extracting at least some of the one or more features using natural language processing comprises performing natural language processing across multiple types of the medical information.

In one aspect, the one or more features include left ventricle ejection fraction and/or a value associated with one or more comorbidities of the patient. In one aspect, the one or more features further include one or more of diagnosis information. In one aspect, the one or more features further include one or more of at least one cardiac value, one or more angiography values, and one or more features of heart disease.

In one aspect, the method further comprises determining whether the patient satisfies one or more inclusion criteria and/or one or more exclusion criteria, and classifying the patient to generate a patient classification comprises generating the patient classification based, at least in part, on whether the patient satisfies the one or more inclusion criteria and/or the one or more exclusion criteria. In one aspect, classifying the patient comprises determining that the patient is not eligible for a Protected PCI® when the patient does not satisfy at least one of the one or more inclusion criteria and/or when the patient satisfies at least one of the one or more exclusion criteria. In one aspect, at least one of the one or more inclusion criteria are based, at least in part, on the extracted one or more features. In one aspect, the one or more inclusion criteria include at least one criterion based on left ventricle ejection fraction.

In one aspect, classifying the patient comprises providing as input to at least one model, the extracted one or more features, wherein the patient classification is generated based, at least in part, on an output of the at least one model. In one aspect, the at least one model is a trained statistical model. In one aspect, the method further comprises receiving data indicating whether the patient received Protected PCI® or would have qualified for Protected PCI®, and updating the at least one model based, at least in part, on a comparison of the received data and the generated patient classification. In one aspect, the at least one model is configured to output a score, and wherein the patient classification is generated based, at least in part, on the score output from the at least one model. In one aspect, the method further comprises determining whether the score output from the at least one model is above a threshold value, and classifying the patient as eligible for a Protected PCI® when it is determined that the score is above the threshold value.

In one aspect, extracting one or more features comprises applying feature-specific extraction logic to the received medical information to extract the one or more features. In one aspect, applying feature-specific extraction logic comprises selecting a procedure report from the received medical information, searching the selected procedure report for one or more keywords, and extracting a feature value for a particular keyword based on identification of the one or more keywords in the selected procedure report. In one aspect, the method further comprises identifying multiple keywords of the one or more keywords in the selected procedure report, and selecting, based on precedence information associated with the multiple keywords in the feature-specific extraction logic, a particular keyword of the multiple keywords, and extracting a feature value for the one or more features comprises extracting the feature value for the particular keyword. In one aspect, the method further comprises determining that the particular keyword is associated with an absolute feature value, and extracting the absolute feature value as the feature value. In one aspect, the method further comprises determining that the particular keyword is associated with a range of values, and extracting the feature value as an average value from the range of values. In one aspect, the method further comprises determining that the particular keyword is associated with text, accessing a lookup table that includes a mapping of words to values, and extracting the feature value based, at least in part, on words in the text associated with the particular keyword and the mapping in the lookup table.

In one aspect, the medical information for the patient is first medical information received at a first time and the method further comprises receiving second medical information for the patient at a second time after the first time, reclassifying the patient the patient with regard to the patient's eligibility for a protected PCI based, at least in part, on the received second medical information, to generate an updated patient classification, and outputting an indication of the updated patient classification. In one aspect, receiving the second medical information comprises receiving the second medical information from an electronic health record of the patient. In one aspect, receiving the second medical information comprises receiving the second medical information via a user interface provided by a computer-implement system. In one aspect, the second medical information includes at least one updated value for the one or more features extracted from the first medical information. In one aspect, the method further comprises extracting one or more updated features from the second medical information, and the reclassifying is performed based, at least in part, on the one or more updated features.

In one aspect, the method further comprises providing a user interface configured to display values for the one or more features, receiving user input via the user interface to change one or more of the values for the one or more features, simulating classification of the patient with regard to the patient's eligibility for a protected PCI based, at least in part, on the changed one or more values, to generate a simulated patient classification, and displaying, on the user interface, the simulated patient classification.

In some embodiments, a computer-implemented system for identifying whether a patient is eligible for a protected percutaneous coronary intervention (PCI) is provided. The system comprises at least one hardware computer processor and at least one non-transitory computer readable medium encoded with a plurality of instructions that, when processed by the at least one hardware computer processor perform a method. The method comprises extracting one or more features from medical information for the patient, classifying the patient with regard to the patient's eligibility for a Protected PCI® based, at least in part, on the extracted one or more features, to generate a patient classification, and outputting an indication of the patient classification.

In some embodiments, at least one non-transitory computer readable medium encoded with a plurality of instructions that, when processed by the at least one hardware computer processor perform a method. The method comprises extracting one or more features from medical information for the patient, classifying the patient with regard to the patient's eligibility for a Protected PCI® based, at least in part, on the extracted one or more features, to generate a patient classification, and outputting an indication of the patient classification.

### BRIEF DESCRIPTION OF DRAWINGS

FIG. 1 illustrates a flowchart of a process for predicting eligibility of a patient for a Protected PCI® in accordance with some embodiments of the present technology;

FIG. 2 schematically illustrates a plurality of features that may be used to generate a hemodynamics compromise score in accordance with some embodiments of the present technology;

FIG. 3 illustrates a flowchart of a process for predicting eligibility of a patient for a Protected PCI® in accordance with some embodiments of the present technology;

FIG. 4 illustrates a flowchart of a process for classifying a patient with regard to the patient's eligibility for a Protected PCI® in accordance with some embodiments of the present technology;

FIG. 5 schematically illustrates two classification techniques for classifying a patient with regard to the patient's eligibility for a Protected PCI® in accordance with some embodiments of the present technology;

FIG. 6 schematically illustrates two classification techniques for classifying a patient with regard to the patient's eligibility for a Protected PCI® in accordance with some embodiments of the present technology;

FIG. 7 schematically illustrates a workflow for classifying a patient with regard to the patient's eligibility for a Protected PCI® when working with an interventional cardiologist in accordance with some embodiments of the present technology;

FIG. 8 schematically illustrates a workflow for classifying a patient with regard to the patient's eligibility for a Protected PCI® when working with a cardiologist in accordance with some embodiments of the present technology;

FIG. 9 schematically illustrates how a model/algorithm used to classify patients in accordance with some embodiments of the present technology may be updated based on a comparison of actual values and predicted values output from the model/algorithm;

FIG. 10 illustrates a flowchart of a process for extracting feature values from electronic medical information in accordance with some embodiments of the present technology; and

FIG. 11 illustrates a flowchart of a process for updating a patient classification in accordance with some embodiments of the present technology.

### DETAILED DESCRIPTION

As is known, Percutaneous Coronary Interventions (PCIs) may be used to open a patient's blocked coronary artery, such as a patient suffering from coronary artery disease. Unfortunately, using conventional risk factor analyses, a significant portion of patients who would benefit from PCI are not qualified even for a high-risk PCI owing to major risk factors. The inventors have recognized and appreciated that such patients may qualify for a PCI if, during the procedure, the patient receives mechanical circulatory support (e.g., via a percutaneous mechanical heart pump) to temporarily support the heart during the procedure and ensure that blood flow is being maintained to critical organs. Such a procedure may be referred to as “Protected PCI®” in that the heart pump serves to protect the patient's heart during the PCI. To this end, some embodiments are directed to data-driven techniques to identify patients that may be candidates for Protected PCI®, for example, despite having higher risk factors that would ordinarily exclude them from high-risk PCI. Predicting which patients will benefit from use of a mechanical heart pump during a PCI, will enable those patients to benefit from having a PCI, thereby improving patient care and surgical outcomes.

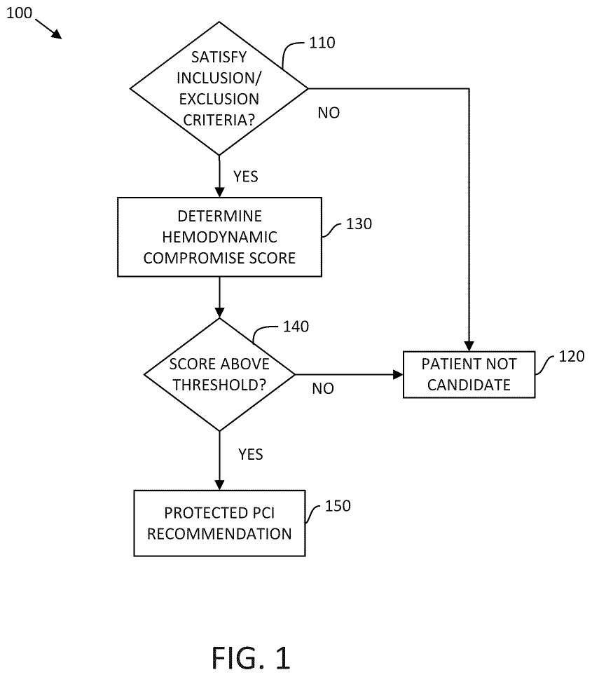

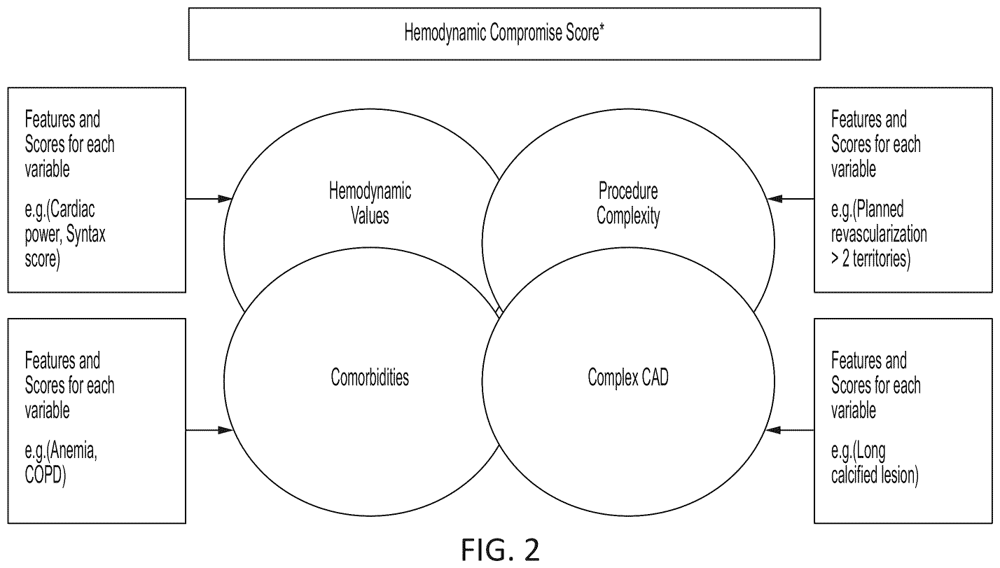

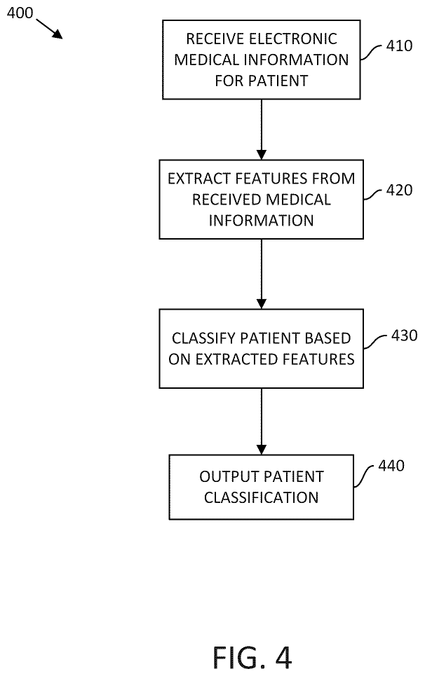

FIG. 1 illustrates a flowchart of a process 100 for identifying patients for Protected PCI® in accordance with some embodiments of the present technology. In act 110, it is determined whether the patient satisfies one or more inclusion and/or exclusion criteria to be considered as a possible candidate for Protected PCI®. Any suitable inclusion/exclusion criteria may be used, examples of which are provided herein. In some embodiments of the present technology, only inclusion criteria are considered in act 110 and exclusion criteria are not considered. If it is determined in act 110 that the patient does not satisfy the inclusion/exclusion criteria, process 100 proceeds to act 120 where it is determined that the patient is not a suitable candidate for Protected PCI®. If it is determined in act 110 that the patient satisfies the inclusion/exclusion criteria, process 100 proceeds to act 130, where a hemodynamic compromise score is determined. In some embodiments, the hemodynamic compromise score may be determined using one or more trained models (e.g., one or more machine learning models) configured to take as input one or more values or features extracted from medical information (e.g., electronic health records, laboratory reports, medical imaging reports, etc.) and output the hemodynamic compromise score. Non-limiting examples of medical information that may be used to determine the hemodynamic compromise score are schematically shown in FIG. 2. Additional types of medical information that may be used to determine the hemodynamic compromise score are described in further detail herein in connection with FIGS. 4-6. Any suitable model(s) may be used to determine the hemodynamics compromise score, and embodiments of the present technology are not limited in this respect.

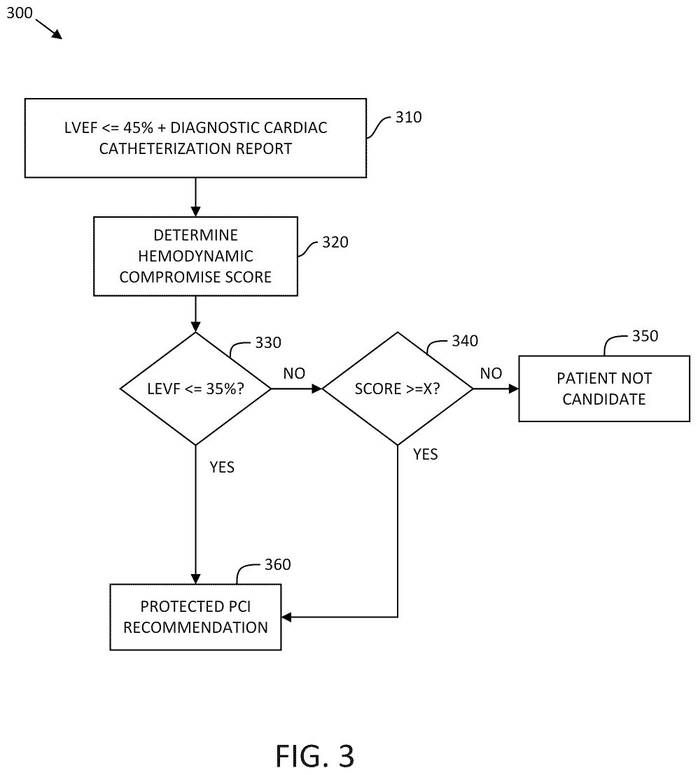

Process 100 then proceeds to act 140, where it is determined whether the determined hemodynamics compromise score is above a threshold value. If it is determined in act 140 that the score is not above the threshold value, process 100 proceeds to act 120, where it is determined that the patient is not a candidate for Protected PCI®. If it is determined in act 140 that the score is above the threshold value, process 100 proceeds to act 150, where the patient is identified as a candidate for Protected PCI®. As described in connection with process 300 shown in FIG. 3, other criteria in addition to determining whether the score is above a threshold may be considered when determining whether the patient is identified as a candidate for Protected PCI®.

In some embodiments, the protected PCI® recommendation determined in act 150 is provided to a healthcare professional (e.g., a physician, such as a cardiologist or an interventional cardiologist, a medical assistant, and/or a health care coordinator). The protected PCI® recommendation may be provided in any suitable way. For instance, an indicator representing the protected PCI® recommendation may be automatically included in the patient's electronic health record. In some embodiments, the indicator may include a textual alert (e.g., “Protected PCI Recommended”). In other embodiments, the indicator may include a visual indicator, such as a green-red indicator system used to provide the Protected PCI® recommendation to the healthcare professional, where “green” signifies that the patient is a good candidate for Protected PCI® and “red” signifies that the patient is not a good candidate for Protected PCI®. In some embodiments, the techniques described herein may be used to analyze a plurality of patients' medical information at a medical facility (e.g., a hospital) to identify and/or prioritize patients for Protected PCI®, and a summary report of those patients most suitable for Protected PCI® may be provided to a medical professional (e.g., a physician) to facilitate clinical decisions regarding the patients' care.

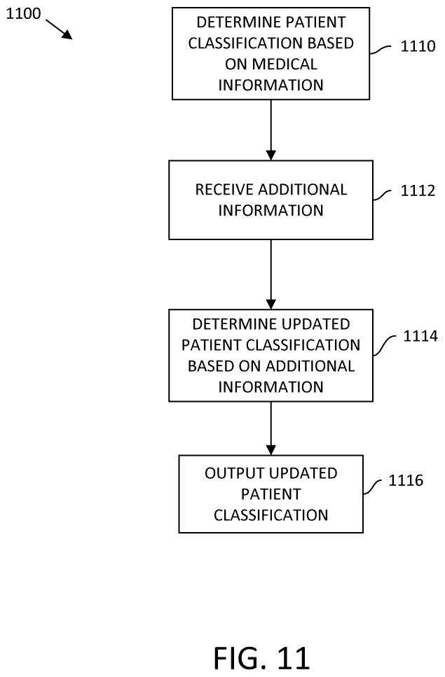

In some embodiments, feedback provided by one or more medical professionals based on the Protected PCI® recommendations may be used to improve the accuracy of the model(s) used to determine the hemodynamic compromise score. For instance, if the medical professional agrees with the recommendation that a patient is a good candidate for Protected PCI® (or alternatively not a good candidate for Protected PCIS), that information may be used to update (e.g., retrain) the model(s) used to determine the hemodynamic compromise score to further improve the accuracy of the model based on the feedback. In some instances, this feedback may be provided to the patient's EHR, which is configured to transmit the feedback to the system. In such instances, the EHR also may be configured to monitor whether or not the doctor has picked a patient for follow up, whether the follow up happened, whether the patient wanted to wait, and/or did the procedure take place. Again, one or more of these parameters may be used to retrain the model. They also may be integrated into the score (see, e.g., the discussion with respect to FIG. 11 in which reclassification of a patient may be enabled via the system), and/or into patient support. As will be appreciated, in other instances, the feedback may be provided to a computer-implemented (or mobile device implemented) system performing the classification. In such instances, the system may be configured to transfer such data to the patient's EHR.

FIG. 3 illustrates a flowchart of a process 300 for identifying patients for Protected PCI® in accordance with some embodiments of the present technology. In the example process shown in FIG. 3, particular features and values are considered for process 100 to determine whether a patient should be identified as a candidate for Protected PCI®. In act 310, the inclusion criteria may include having a left ventricle ejection fraction (LVEF) less than or equal to 45% and having a diagnostic cardiac catheterization report in their electronic health record. No exclusion criteria are specified. If it is determined that the patient satisfies these inclusion criteria, process 300 proceeds to act 320, where a hemodynamic compromise score is determined for the patient. As discussed for process 100, the hemodynamic compromise score may be determined as the output of one or more models that take as input, features or values based on medical information for the patient. The input features or values provided as input to the model(s) may relate to the inclusion criteria considered in act 310 and/or may be unrelated to the inclusion criteria.

Process 300 then proceeds to act 330, where it is determined whether the LVEF of the patient is less than or equal to 35%. If it is determined in act 330 that LVEF is less than or equal to 35%, process 300 proceeds to act 360, where the patient is identified as a candidate for Protected PCI®. If it is determined in act 330 that the patient's LVEF is not less than or equal to 35% (i.e., LVEF is 35%-45%), process 300 proceeds to act 340, where it is determined whether the hemodynamic compromise score is above a threshold value X. If it is determined in act 340 that the score is less than the threshold X, process 300 proceeds to act 350, where the patient is identified as not being a suitable candidate for Protected PCI®. If it is determined in act 340 that the score is greater than the threshold X, process 300 proceeds to act 360, where the patient is identified as a candidate for Protected PCI®.

FIG. 4 is a flowchart of a process 400 for classifying a patient as being a good/poor candidate for a Protected PCI® procedure based on medical information in accordance with some embodiments of the present technology. In act 410, electronic medical information for the patient is received. The electronic medical information may include, but is not limited to, an electronic health record (EHR), laboratory reports, medical procedure reports (e.g., electrocardiograph reports), physician notes, and medical imaging reports. Process 400 then proceeds to act 420, where one or more features are extracted from the received medical information. The one or more extracted features may include, but are not limited to, diagnosis information, heart function values (e.g., LVEF, cardiac power), comorbidities (e.g., anemia, chronic obstructive pulmonary disease (COPD), hypertension), angiography information and heart disease descriptors (e.g., long calcified lesion and mitral regurgitation). The received medical information may include structured data (e.g., organized based on an ontology, for example, using labels, fields, or other metadata associated with the data that can be used for feature extraction) and/or unstructured data (e.g., physicians' notes entered into a free text field in an electronic form).

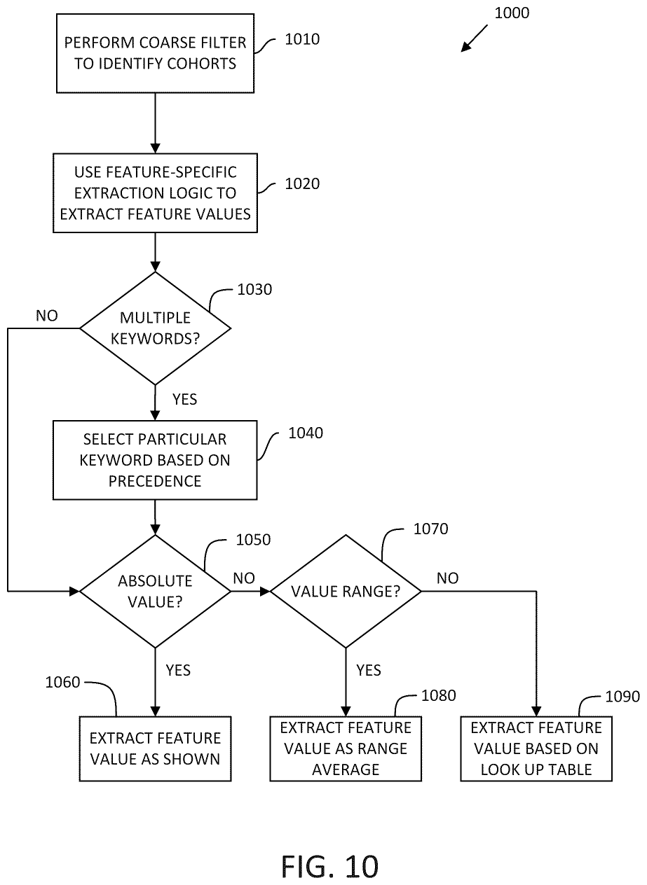

In some embodiments of the present technology, at least some of the one or more features are extracted using natural language processing (NLP) techniques. Such NLP techniques may be used to analyze text in the received medical information to infer information determined as features. NLP may be used to analyze individual types of medical information (e.g., medical reports) and/or may be used across multiple types of medical information to extract the one or more features. In some instances, a first set of features may be extracted using one or more manual techniques and a second set of features may be extracted using automated (e.g., NLP) techniques. Automated techniques for extracting one or more features from a patient's electronic medical information (e.g., an electronic health record) are described in more detail herein with regard to FIG. 10.

Process 400 then proceeds to act 430, where the patient is classified (e.g., as being a good candidate for Protected PCI®) based, at least in part, on the extracted one or more features. The extracted features may be provided, for example, as input to a model (e.g., a trained machine learning model) trained to output a patient classification, or may be provided as input to an algorithm that weights the features to determine a score on which a classification for the patient is based. Process 400 then proceeds to act 440, where the patient classification determined in act 430 is output. The patient classification may be output in any suitable way, examples of which are described above in connection with act 150 of process 100 shown in FIG. 1. For instance, a visual indicator (e.g., red, yellow, green) may be associated with the patient to indicate the classification. Alternatively, process 400 may be performed for each of a plurality of patients in a medical facility, and a list of patients classified as being most likely to receive benefit from a Protected PCI® may be output in act 440.

In some embodiments, a patient may be flagged for additional follow-up (e.g., if the patient is a “borderline” case that is close to being recommended for Protected PCI®. As described herein in connection with FIG. 11, in some embodiments, patients initially classified as not recommended for Protected PCI® may be reclassified as the patient's medical status changes over time. For instance, as additional medical information associated with the patient becomes available (e.g., by being entered in the patient's electronic health record and/or by being entered directly into a computer-implemented (or mobile device implemented) system configured to output the patient classification) a patient initially classified as not recommended for Protected PCI® may be reclassified as recommended for Protected PCI® based on intervening medical data (e.g., additional cardiac catheterization laboratory reports, additional blood work data, etc.) that may be considered during reclassification. In this way, patients' classification status at a healthcare facility may be tracked over time to dynamically identify patients that may benefit from Protected PCI® procedures.

As will be appreciated, a patient also may be flagged for additional follow-up if they present as an interesting case (e.g., could be a patient who is classified as recommended for Protected PCI®) and a physician requires additional information before recommending a procedure to their patient. As with the above, once additional information has been received, the patient may be again reclassified (e.g., with a similar or different classification), and the physician may make a recommendation to their patient based upon the classification.

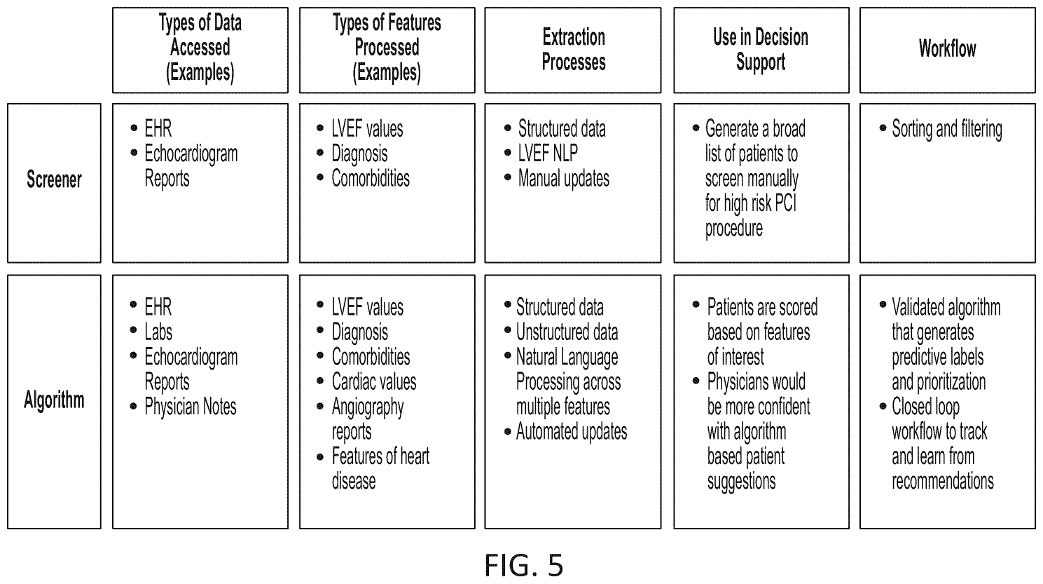

In some embodiments of the present technology, different classification techniques may be used at different healthcare systems and/or multiple classification techniques may be used at a single healthcare system. FIG. 5 schematically illustrates two such techniques—a “screener” classification technique configured to analyze primarily (or only) electronic health records (EHRs) to identify patients at a health center for further manual screening to assess eligibility for Protected PCI®, and an “algorithm” classification technique that has a higher level of complexity compared to the screener classification technique in that patients are classified using models or algorithms based on extracted features, as described for example, in connection with the processes of FIGS. 1, 3 and 4. The screener technique may be less complex than the algorithm technique in that one or more of the following may be true: the screener technique may operate on limited patient data (e.g., only EHR data); the screener technique may provide simpler feature extraction processes than the algorithm technique; and the output of the screener technique may be less complex and/or predictive than the output of the algorithm technique, such that further follow up (either by a person or another classification process) may be required for the screener technique, but not necessarily for the algorithm technique.

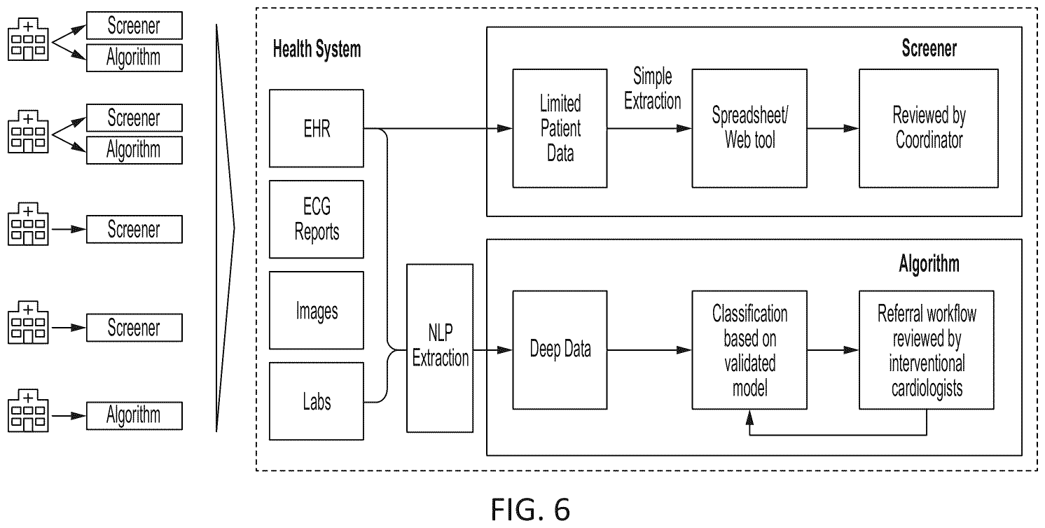

FIG. 6 schematically illustrates that different healthcare systems (e.g., hospitals, medical clinics, etc.) may use one or more classification techniques to identify patients who would benefit from a Protected PCI® in accordance with the methods described herein. For instance, continuing with the two example classification techniques described in FIG. 5, some healthcare systems may employ the “screener” classification technique, other healthcare systems may employ the “algorithm” classification technique, whereas yet other healthcare systems may employ both techniques or combinations thereof. As shown in FIG. 6, the screener classification technique may have a simpler workflow and may be advantageous to use in healthcare systems that have limited patient data (though its use is not so limited). For instance, rural clinics or other healthcare facilities without laboratory and/or medical imaging facilities may be more well-suited to using the screener classification technique. However, it should be appreciated that use of the less complex screener technique is not so limited. For instance, a large healthcare system may employ the screener classification technique to identify a subset of patients who may benefit from a Protected PCI® procedure. The medical information for the subset of patients identified by the screener classification technique may then be further analyzed using the algorithm classification technique (or some other classification technique) to refine the subset of patients to identify a smaller subset of patients eligible for a Protected PCI®. It should be appreciated that some healthcare systems may only employ one type of classification technique (e.g., only the screener technique, the algorithm technique, or some other technique not shown).

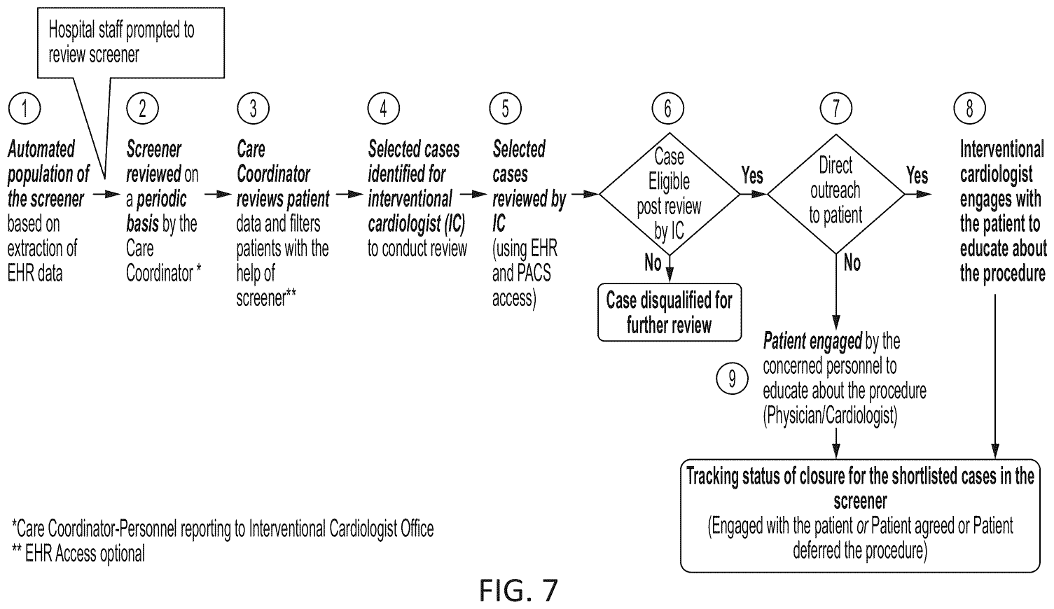

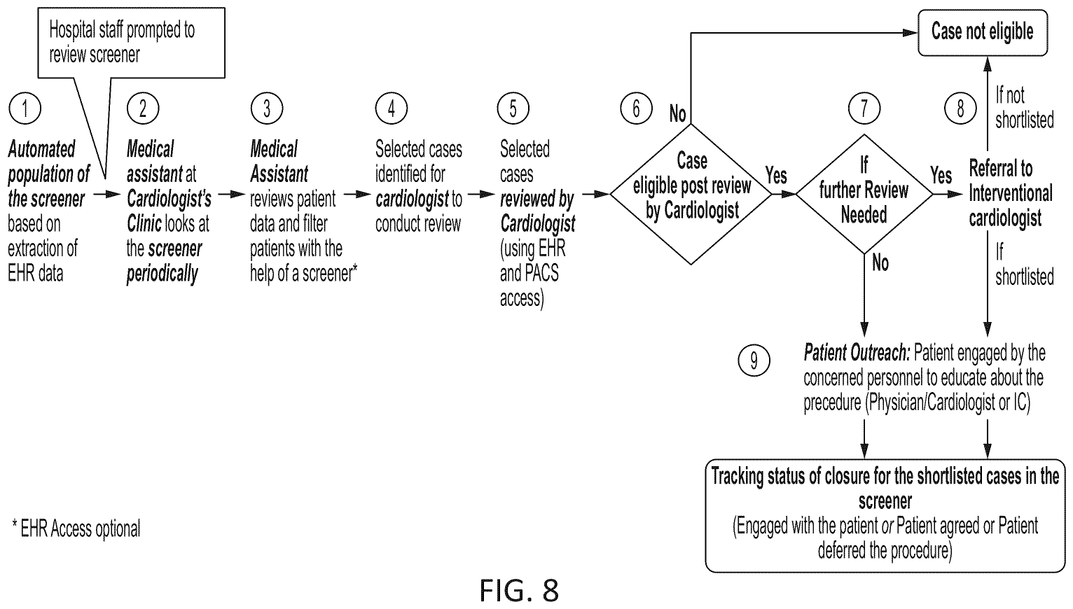

FIGS. 7 and 8 illustrate example workflows for classifying a patient as being eligible or not eligible for a Protected PCI® using the techniques described herein. In the example workflow shown in FIG. 7, a care coordinator may be working with an Interventional Cardiologist (IC). As an overview of the workflow, the results of the screener classification technique may be reviewed by the care coordinator from the IC office. Based on that review a subset of “shortlisted” cases may be referred to the IC for review, after which they are reviewed by the IC. In some embodiments, the shortlisted cases may be determined based, at least in part, on an automated analysis of data extracted from an electronic health record and/or other electronic medical information for a patient, examples of which are described herein. In some embodiments, automatically analyzing the extracted data includes associating one or more scores with a set of extracted data. For instance, the extracted data may be associated with one or more categories and a score may be applied to each category of extracted data. Non-limiting examples of categories associated with extracted data include, but are not limited to, gender, age, family history of cardiac disease, a LVEF value, a LVEF chronology, comorbidities (e.g., cardiac morbidities (e.g., successful resuscitation after cardiac event), non-cardiac major comorbidities (e.g., end stage renal disease (ESRD), peripheral vascular disease (PVD), other comorbidities), comorbidity combinations, presence of one or more diseases (e.g., coronary artery disease, left main disease, and/or chronic total occlusion), findings from an ECHO (e.g., TTE, TEE, stress), whether or not a cardiac surgeon or interventional cardiologist has been consulted, number of diagnostic catheterizations, number of follow-up hospital readmissions, heart failure medications, other cardiac medications, or whether or not the patient has undergone a prior coronary artery bypass graft (CABG), a PCI procedure, an open-heart procedure, and/or had a procedure to implant a cardiac device. In some embodiments, the score assigned to a category of extracted data depends, at least in part, on the value of the extracted data. For example, for an extracted LVEF value, a first score may be used if the LVEF<=40% and a second score may be used if the LVEF is between 41% and 50%. In some embodiments, the score associated with extracted data may be selected to represent a level of risk associated with the particular data. For instance, a higher score may be assigned to each category of data having values that correspond to higher risk factors for a patient. In some embodiments, a cumulative score (e.g., across multiple categories) may be provided for review. In some embodiments, the cumulative score may be determined by adding the scores associated with each category. In some embodiments, data associated with each category may be weighted evenly, such that the cumulative score is determined by addition of the individual category scores. In other embodiments, data associated with one or more categories may be weighted higher than other categories, such that the cumulative score represents a weighted sum of the individual category scores. In this regard, a first category may be weighted more heavily than a second category if the first category may correlate to a higher risk factor for the patient than the second category.

Information relating to the scores may be provided to the care coordinator in any suitable way. For instance, the care coordinator may be provided with information in a user interface that provides an indication of the score and/or describes how the score was calculated based on the extracted data. For the shortlisted cases, the IC may engage with the patient or refer identified cases to a physician or cardiologist for patient outreach. Based on a review of the results of the screener classification technique, a user may interact with a user interface to change one or more aspects of the scoring, and such changes may be reflected in the shortlist of cases identified during the workflow. In some embodiments, a status assigned to a particular patient may be additionally or alternatively be used to update the shortlist of cases identified during the workflow.

In the example workflow shown in FIG. 8, a medical assistant may be working with a cardiologist. As an overview of the workflow, the results of the screener classification technique may be reviewed by the medical assistant from the cardiologist's clinic. Based on that review, a subset of “shortlisted” cases may be referred to the cardiologist for review, after which they are reviewed by the cardiologist. As with the workflow in FIG. 7, the shortlisted cases may be determined, at least, in part, on an automated analysis of data extracted from an electronic health record and/or other electronic medical information for a patient, examples of which are described herein. For the shortlisted cases, the cardiologist may engage with the patient or refer the patient to an IC for further view and/or opinion. Based on a review of the results of the screener classification technique, a user (e.g., the cardiologist) may interact with a user interface to change one or more aspects of the scoring, and such changes may be reflected in the shortlist of cases identified during the workflow. In some embodiments, a status assigned to a particular patient may be additionally or alternatively be used to update the shortlist of cases identified during the workflow.

As will be appreciated, for the workflows in both FIGS. 7 and 8, patients may be listed as “borderline” in some instances, as described with respect to FIG. 11, and reclassified after additional information is made available. In such instances, the classification may be updated after such additional information is made available, with the new classification being relayed to the hospital staff.

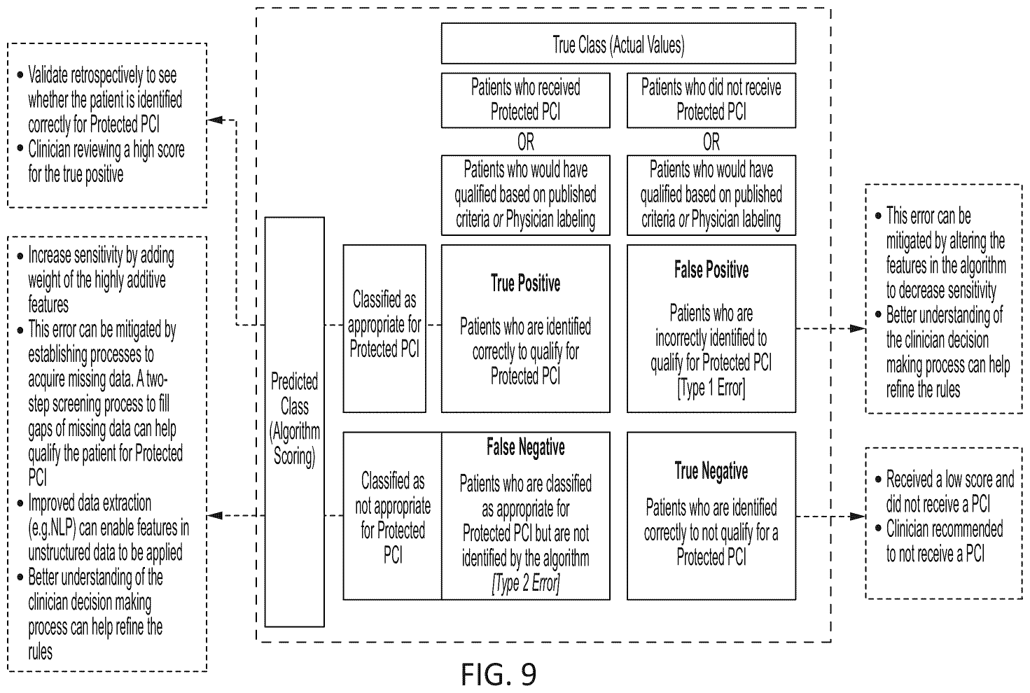

As described herein, for embodiments of the present technology that employ models or algorithms in which extracted features may be weighted to determine the patient classification, the models/algorithms may be updated (e.g., retrained or tuned) based on feedback about whether the classification provided by the model/algorithm agreed or disagreed with an assessment of a healthcare professional reviewing the patient's eligibility for a Protected PCI®. FIG. 9 schematically illustrates a comparison of predictions or recommendations output by the model/algorithm, and actual values, that is patients who received/did not receive Protected PCI® or would/would not have qualified based on published criteria or physician labeling. FIG. 9 further illustrates example ways in which the model/algorithm may be tuned to mitigate false positives and false negatives by applying the feedback to tune the model/algorithm.

As shown, the model/algorithm performs well when there is a true positive or a true negative, that is the output of the model/algorithm and the actual values agree. False negatives (patient received or qualified for Protected PCI®, but model/algorithm did not identify patient as eligible for Protected PCI®) may occur, for example, when the model/algorithm is not sensitive enough to (e.g., does not weight enough) the features that most strongly correlate with a Protected PCI® determination. In such instances, the weights for those features may be adjusted accordingly, to improve the predictions output from the model/algorithm. Other aspects of the model/algorithm may also be updated, as noted in FIG. 9. False positives (patient did not receive or was not eligible for Protected PCI®, but model/algorithm identified patient as eligible for Protected PCI®) may occur, for example, if the model/algorithm is too sensitive to (e.g., weights too heavily) certain features. In such instances, the weights for those features may be adjusted accordingly, to improve the predictions output from the model/algorithm.

As described herein (for example, in connection with act 420 of process 400 shown in FIG. 4), some embodiments may relate to extracting one or more features from electronic medical information to facilitate patient classification. The inventors have recognized and appreciated that variability in how information is represented and described in electronic medical information may complicate the extraction of one or more features if, for example, simple keyword searching or medical task code-based extraction is used. For example, the medical diagnosis of Coronary Artery Disease (CAD) may not be consistently indicated in patients' electronic health records (EHRs). If an extraction technique for determining whether a patient has CAD was configured to search the patient's EHR only for the presence/absence of a particular medical task code corresponding to CAD, for example, some patients having characteristics of CAD, but without a clear diagnosis using the particular task code in their EHR may be missed. Some embodiments herein may extract feature(s) (e.g., whether a patient has CAD) from electronic medical information by examining additional data elements, such as medications and/or procedures related to the feature (e.g., procedures related to CAD).

FIG. 10 illustrates a process 1000 for multi-granular extraction of one or more features from electronic medical information, in accordance with some embodiments. In act 1010, electronic health information for a plurality of patients may be analyzed to separate the patients into a plurality of cohorts, an example of which is shown in Table 1.

TABLE 1
Example cohorts for a patient population

| | Cohort 1 | | Cohort 2 | | Cohort 3 | | | | |
|---|---|---|---|---|---|---|---|---|---|
| | Patients having | | Patients having | | Cohort 3A: Patients | | Cohort 3B: Patients with | | |
| | undergone | | undergone high | | with LEVF <= 35% | | LEVF <= 35%, no PCI | | Cohort 4 |
| | PCI | | risk PCI | | and no PCI | | and CAD diagnosis | | All Patients |
| | | % | | % | | % | | % | Total Patient |
| | Count | Cohort 4 | Count | Cohort 4 | Count | Cohort 4 | Count | Cohort 4 | Count |
| Unique | 6000 | 0.17% | 60 | 0.02% | 2000 | 0.06% | 1000 | 0.03% | 3,500,000 |
| Patient | | | | | | | | | |
| Count | | | | | | | | | |

As shown in the example cohorts of Table 1, for cohort 3 in which patients had a low LEVF value (e.g., <35%) and did not have a PCI procedure, only ⅓ of those patients (the patients in cohort 3B) had a CAD diagnosis described in their electronic medical information even though all of the patients in cohort 3 may be considered as possible candidates for a Protected PCI® procedure due to their low LEVF values. Some embodiments include techniques for using additional information (e.g., medication information, procedure information) in a patient's electronic medical information to determine whether the patient is associated with a particular feature (e.g., whether the patient has coronary artery disease) when such an association may not be explicit in the patient's electronic medical information. For instance, in the example cohorts shown in Table 1, additional electronic medical information (e.g., procedure reports) for the 2000 patients in Cohort 3A may be analyzed to determine whether some or all of the patients have CAD (and thus may be eligible for Protected PCI®) despite not having an explicit CAD diagnosis stated in their medical record. The additional electronic medical information may include, but is not limited to, electrocardiographs, transthoracic echocardiograms (TTEs), cardiovascular stress echoes, nuclear stress tests, cardiovascular stress tests, Doppler ultrasound tests, coronary diagnostic angiograms and cardiac catheterization reports. The additional electronic medical information may include structured data and/or unstructured data.

Returning to process 1000, after separation of patients into a plurality of cohorts, process 1000 may proceed to act 1020, where feature-specific extraction logic is used to extract one or more features of interest from the additional electronic medical information. For instance, the feature-specific extraction logic for the feature “LVEF” may search for the presence of a TTE in the patient's electronic medical information, and if found, information associated with LVEF may be extracted from the TTE.

The information associated with the particular feature may be extracted from the patient's electronic medical information in any suitable way. In some embodiments, extraction is performed using natural language processing. For instance, one or more reports or other documents as specified in the feature-specific logic may be searched for one or more keywords to identify the information associated with the feature to be extracted. When the feature-specific logic is configured to search for multiple keywords, the feature-specific logic may assign a precedence to the multiple keywords such that if multiple keywords are present in the electronic medical information, the keyword assigned a higher precedence will be selected for extraction of its corresponding value. Accordingly, process 1000 may proceed to act 1030, where it is determined whether the electronic medical information (e.g., a TTE) includes multiple keywords. If it is determined in act 1030 that the electronic medical information includes multiple keywords, process 1000 may proceed to act 1040, where a particular keyword is selected based on precedence information associated with the multiple keywords. For instance, as described above, the keyword with the higher precedence may be selected. If it is determined in act 1030 that the electronic medical information does not include multiple keywords or after selection of a particular keyword in act 1040, process 1000 may proceed to act 1050, where it is determined whether the selected keyword is associated with an absolute value for the feature. If it is determined that the keyword is associated with an absolute value for the feature, process 1000 may proceed to act 1060, where the value for the feature associated with the keyword is extracted as the feature value. Otherwise, process 1000 may proceed to act 1070, where it is determined whether the keyword is associated with a range of values. If it is determined in act 1070 that the keyword is associated with a range of values, process 1000 may proceed to act 1080, where the feature value is determined as an average value from the range of values associated with the keyword. Otherwise, process 1000 may proceed to act 1090, where a look up table is used to determine a value for the feature. For instance, the electronic medical information may include one or more words (e.g., normal, excellent, lower limit of normal, poor, severe, abnormal, etc.) that qualitatively describe the value for the feature. The extracted value for the feature may be based on a look up table that maps one or more of these words to values (e.g., based on ranges specified in medical literature).

Acts 1020-1090 may be repeated for multiple features of interest to extract feature values from a patient's electronic medical information for the multiple features. The extracted feature values may then be used, at least in part, to classify the patient (e.g., as a patient for Protected PCI®) as described herein with regard to act 430 in process 400 of FIG. 4.

The inventors have recognized and appreciated that patients initially classified as not recommended for a Protected PCI® may later be classified as a patient recommended for a Protected PCI® due to intervening events between when the patient was initially classified and the later point in time. Accordingly, the inventors have appreciated the benefit of a system in which a patient classification (e.g., whether a patient is a candidate for a Protected PCI®) may be updated as additional information (e.g., additional electronic medical information) associated with the patient becomes available. For instance, a physician or other healthcare provider may input additional medical information into a user interface associated with an electronic health record for the patient, and the patient classification may be updated based on the additional medical information. In other instances, the physician may order one or more tests that may be performed and later added to the patient's electronic health record. In still other instances, a physician or other healthcare provider may enter additional information directly into a computer-implemented (or mobile device implemented) system performing the patient classification. In such instances, updating the patient classification over time when new information associated with the patient becomes available may enable longitudinal tracking of patient's classification status. For instance, “borderline” patients initially classified as not being candidates for a Protected PCI® may have their status reevaluated based on the additional information such that they may be reclassified as a candidate for a Protected PCI® based on the additional information.

FIG. 11 illustrates a process 1100 for updating a patient classification based on additional information, in accordance with some embodiments of the present technology. In act 1110 a patient may be classified (e.g., as being a good/poor candidate for Protected PCI®) based, at least in part, on medical information associated with the patient. For example, the patient may be classified as a poor candidate for Protected PCI® based, at least in part, on extracted feature(s) from the patient's electronic health record, examples of which are described herein. Process 1100 may then proceed to act 1112, where additional information associated with the patient is received. For instance, a physician may interact with a user interface of a computer system configured to implement one or more of the classification techniques described herein (e.g., a screener classification technique, an algorithm classification technique) to input at least some of the additional information. Alternatively, at least some of the additional information may be entered into the patient's electronic health record (EHR) and the additional information may be provided from the EHR as input to a classification technique. In some embodiments, one or more of the classification techniques described herein may be integrated with an EHR (e.g., as a module or plugin associated with the EHR), such that when additional information is entered into the EHR, and updated classification may be generated automatically and/or in response to a user request to generate the patient classification. In such embodiments, receiving additional information in act 1112 of process 1100 may be contemporaneous with receiving the additional information as it is entered into the EHR. Act 1112 also may be contemporaneous with other acts of process 1100 (e.g., act 1114 and 1116, as described below).

In instances in which the classification system is implemented as part of an EHR, it will be appreciated that the plug-in also may be configured to perform the calculation after a certain prescribed period of time (e.g., every 5, 10, 15, 20 minutes) after new patient information is added to the patient record.

After receiving additional information in act 1112, process 1100 may proceed to act 1114, where an updated patient classification may be determined based, at least in part, on the additional information. For instance, the additional information may be provided as input to one or more of the classification techniques described herein to determine the updated patent classification. The additional information may be considered by the classification technique(s) in any suitable way. For example, the additional information may be used replace some of the information used to generate the initial patient classification (e.g., the patient classification determined in act 1110). As a specific example, an updated LVEF value for the patient may be extracted form a new TTE that was not available when the initial patient classification was determined, and the updated LVEF value for the patient may be used in place of the LVEF values used during the initial patient classification determination. In some embodiments, the additional information may augment the data and/or features that were used in the initial patient classification data without replacing any particular values. Process 1100 may then proceed to act 1116, where the updated patient classification may be output. As discussed in connection with process 400 in FIG. 4, the updated patient classification may be output in any suitable way, examples of which are described above in connection with act 150 of process 100 shown in FIG. 1. For instance, a visual indicator (e.g., red, yellow, green) may be associated with the patient to indicate the classification. As described with other processes herein, it should be appreciated that process 1100 may be performed for each of a plurality of patients in a medical facility, and a list of patients classified as being most likely to receive benefit from a Protected PCI® may be output in act 1116. In this way, all or a subset of patients at a healthcare facility may be longitudinally tracked to identify a current list of patients that may benefit from a Protected PCI® as new information about the patients at the healthcare facility becomes available for consideration by the classification technique.

In some embodiments, a computer-implemented system configured to execute one or more of the patient classification techniques described herein may present a user interface that enables a physician or other healthcare provider the ability to test “what-if” scenarios by adjusting values of one or more features used to generate a patient classification. For instance, the user interface may display values for a plurality of features extracted from a patient's EHR used to generate a current patient classification for the patient. The healthcare provider may then interact with the user interface to change one or more of the values (e.g., by typing or otherwise inputting a new value into a field of the user interface) displayed on the user interface, and the patient may be reclassified (e.g., as a simulation) based, at least in part, on the changed values. In some embodiments, the user interface may present a slider element to represent values for one or more of the features, and the healthcare provider may change the values by interacting with the slider element. By enabling the healthcare provider to simulate how changes in various feature values affect the patient classification, the healthcare provider may gain a better understanding of how different features contribute to the patient classification determination and may provide insight on particular features to monitor in different patients that if changed may cause the patient to be recommended for a Protected PCI®.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of identifying whether a patient is eligible for a protected percutaneous coronary intervention (PCI), the method comprising:
receiving medical information for a patient;
extracting one or more features from the received medical information;
classifying the patient with regard to the patient's eligibility for a protected PCI based, at least in part, on the extracted one or more features, to generate a patient classification; and
outputting an indication of the patient classification.

2. The method of claim 1, wherein the medical information for the patient includes one or more of an electronic health record, a laboratory report, an electrocardiograph report, and a medical imaging report.

3-9. (canceled)

10. The method of claim 1, further comprising:
determining whether the patient satisfies one or more inclusion criteria and/or one or more exclusion criteria, and wherein
classifying the patient to generate a patient classification comprises generating the patient classification based, at least in part, on whether the patient satisfies the one or more inclusion criteria and/or the one or more exclusion criteria.

11. The method of claim 10, wherein classifying the patient comprises determining that the patient is not eligible for a protected PCI when the patient does not satisfy at least one of the one or more inclusion criteria and/or when the patient satisfies at least one of the one or more exclusion criteria.

12. (canceled)

13. (canceled)

14. The method of claim 1, wherein classifying the patient comprises:
providing as input to at least one model, the extracted one or more features, wherein the patient classification is generated based, at least in part, on an output of the at least one model.

15. (canceled)

16. The method of claim 14, further comprising:
receiving data indicating whether the patient received protected PCI or would have qualified for protected PCI; and
updating the at least one model based, at least in part, on a comparison of the received data and the generated patient classification.

17. The method of claim 14, wherein the at least one model is configured to output a score, and wherein the patient classification is generated based, at least in part, on the score output from the at least one model.

18. The method of claim 17, further comprising:
determining whether the score output from the at least one model is above a threshold value; and
classifying the patient as eligible for a protected PCI when it is determined that the score is above the threshold value.

19. The method of claim 1, wherein the medical information for the patient is first medical information received at a first time, the method further comprising:
receiving second medical information for the patient at a second time after the first time;
reclassifying the patient the patient with regard to the patient's eligibility for a protected PCI based, at least in part, on the received second medical information, to generate an updated patient classification; and
outputting an indication of the updated patient classification.

20. The method of claim 19, wherein receiving the second medical information comprises receiving the second medical information from an electronic health record of the patient or receiving the second medical information via a user interface provided by a computer-implemented system.

21. (canceled)

22. (canceled)

23. The method of claim 19, further comprising:
extracting one or more updated features from the second medical information,
wherein the reclassifying is performed based, at least in part, on the one or more updated features.

24. The method of claim 23, wherein extracting one or more features comprises:
applying feature-specific extraction logic to the received medical information to extract the one or more features.

25. The method of claim 24, wherein applying feature-specific extraction logic comprises:
selecting a procedure report from the received medical information;
searching the selected procedure report for one or more keywords; and
extracting a feature value for a particular keyword based on identification of the one or more keywords in the selected procedure report.

26. The method of claim 25, further comprising:
identifying multiple keywords of the one or more keywords in the selected procedure report; and
selecting, based on precedence information associated with the multiple keywords in the feature-specific extraction logic, a particular keyword of the multiple keywords,
wherein extracting a feature value for the one or more features comprises extracting the feature value for the particular keyword.

27. The method of claim 25, further comprising:
determining that the particular keyword is associated with an absolute feature value; and
extracting the absolute feature value as the feature value.

28. The method of claim 25, further comprising:
determining that the particular keyword is associated with a range of values; and
extracting the feature value as an average value from the range of values.

29. The method of claim 25, further comprising:
determining that the particular keyword is associated with text;
accessing a lookup table that includes a mapping of words to values; and
extracting the feature value based, at least in part, on words in the text associated with the particular keyword and the mapping in the lookup table.

30. The method of claim 1, further comprising:
providing a user interface configured to display values for the one or more features;
receiving user input via the user interface to change one or more of the values for the one or more features;
simulating classification of the patient with regard to the patient's eligibility for a protected PCI based, at least in part, on the changed one or more values, to generate a simulated patient classification; and
displaying, on the user interface, the simulated patient classification.

31. A computer-implemented system for identifying whether a patient is eligible for a protected percutaneous coronary intervention (PCI), the system comprising:
at least one hardware computer processor; and
at least one non-transitory computer readable medium encoded with a plurality of instructions that, when processed by the at least one hardware computer processor perform a method, the method comprising:
extracting one or more features from medical information for the patient;
classifying the patient with regard to the patient's eligibility for a protected PCI based, at least in part, on the extracted one or more features, to generate a patient classification; and
outputting an indication of the patient classification.

32. At least one non-transitory computer readable medium encoded with a plurality of instructions that, when processed by the at least one hardware computer processor perform a method, the method comprising:
extracting one or more features from medical information for the patient;
classifying the patient with regard to the patient's eligibility for a protected PCI based, at least in part, on the extracted one or more features, to generate a patient classification; and
outputting an indication of the patient classification.
